(19) United States
(12) Patent Application Publication
(10) Pub. No.: US 2007/0031339 A1
(43) Pub. Date: Feb. 8, 2007

(54) Contrast agents

(76) Inventors: Oskar Axelsson, Malmo (SE); Ib Leunbach, Malmo (SE); Magnus Karlssov, Malmo (SE)

Correspondence Address: GE HEALTHCARE, INC., IP DEPARTMENT, 101 CARNEGIE CENTER, PRINCETON, NJ, 08540-6231, US

(21) Appl. No.: 10/560,065

(22) PCT Filed: Nov. 6, 2004
(86) PCT No.: PCT/NO04/00364

§ 371 Date: Dec. 8, 2005

(30) Foreign Application Priority Data
Nov. 28, 2003 (NO) 20035294
Oct. 26, 2004 (NO) 20044622

(51) Int. Cl. A61K 49/04

(52) U.S. Cl. 424/9.42; 977/928

(57) ABSTRACT

The present invention relates to particles comprising cores of tungsten or tungsten in mixture with other metallic elements as the contrast enhancing material wherein said core are coated, to pharmaceuticals containing such particles, and to the use of such pharmaceuticals specifically as contrast agents in diagnostic imaging, in particular in X-ray imaging.

The present invention relates to particles and to pharmaceuticals containing such particles where the particles comprise coated cores of the metallic element of tungsten or of tungsten in mixture with other metallic elements as the contrast enhancing material. The invention also relates to the use of such pharmaceuticals as contrast agents in diagnostic imaging, in particular X-ray imaging and to contrast media containing such cores of the metallic element of tungsten or tungsten in mixture with other metallic elements.

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is, for a given spatial resolution, strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials, contrast agents, into the body region being imaged.

Thus in X-ray for example early examples of contrast agents were insoluble inorganic barium salts which enhanced X-ray attenuation in the body zones into which they distributed. More recently the field of X-ray contrast agents has been dominated by soluble iodine containing compounds such as those marketed by Amersham Health AS under the trade names Omnipaque and Visipaque.

Work on X-ray contrast agents having heavy metals as the contrast enhancing element has to a great extent concentrated on aminopolycarboxylic acid (APCA) chelates of heavy metal ions. Recognising that effective imaging of many body sites requires localization at the body sites in question of relatively high concentrations of the metal ions, there have been suggestions that polychelants, that is substances possessing more than one separate chelant moiety, might be used to achieve this. Further work has been concentrated on the use of multinuclear complexes that are complexes wherein the complexed moiety itself comprises two or more contrast enhancing atoms, see Yu, S. B. and Watson, A. D. in Chem. Rev. 1999, 2353-2377. Thus, for X-ray or ultrasound the complexes would comprise two or more heavy metal atoms and for MRI the complex would contain two or more metal atoms with paramagnetic properties.

Yu, S. B. and Watson, A. D. in Chem. Rev. 1999, 2353-2377 discuss use of metal-based X-Ray contrast media. Tungsten powder is noted for use as an X-ray contrast additive in embolic agents used in the treatment and preoperative embolisation of hypervascular tumors. However, they find it likely that general intravascular use of heavy metal complexes is limited by safety concerns and dosage requirements.

It is well known that nano-crystalline tungsten powder is pyrophoric and ignites spontaneously in air. Due to its reactivity tungsten nanoparticles have not found use as pharmaceuticals such as X-ray contrast agents.

Metal conjugate compounds of metallic heavy elements of gold, silver, platinum and palladium are proposed as well as their use as contrast agents such as X-ray contrast agents e.g. in U.S. Pat. No. 5,728,590. Further, U.S. Pat. No. 6,203,778 mentions that particles of an inorganic core of metallic copper, nickel, palladium, gold and silver with an organic coating can be used in an X-ray imaging method.

WO 03/07961 reads in particular on metal nanoparticles for use in enhancing X-ray contrast. The patent application is focussed on gold particles in the nanometer range including particles covalently attached to antibodies. The gold particles are coated with thioglucose to make them more physiological tolerable, other coatings such as glutathione were tried but were found to be less tolerable. Platinum, palladium, thallium, bismuth, osmium, iridium, silver, tungsten, lead, tantalum and uranium are also mentioned as possible alternative metals.

The gold cores of the nanoparticles described in WO 03/07961 have a substantially inert surface and the purpose with the thioglucose coating is not to passivate the surface.

The thioglucose coating of the gold particles is exchangeable and the binding between the surface of the gold particles and the coating is relatively weak. These coated gold particles will hence tend to have a long half-life in the body because of exchange of the ligands in the coating with groups in the tissue e.g. protein sulfhydryl groups. Uncoated gold particles will therefore remain in the blood stream, see e.g. Hostetler, M. J.; Templeton, A. C.; Murray, R. W; “Dynamics of Place-Exchange Reactions on Monolayer-Protected Gold Cluster Molecules” Langmuir, 1999, 15, 3782-3789. The long half-life in the body is not desirable because this could lead to higher toxicity and the long half-life is generally not an advantage in X-ray investigations.

As outlined above, various metals are known from the state of art for use as contrast agents including cores of the elements in its metallic (0) oxidation state. Coated nanoparticles have been proposed for use as X-ray contrast agents. Nanoparticles of the substantially inert metals such as gold, silver, palladium and platinum are preferred for use as pharmaceuticals. Many of the inert metals such as gold, gadolinium, erbium and other rare earth elements are however expensive and less viable for use as commercial contrast agents. Others, such as uranium, are radioactive and hence not suitable as X-ray contrast agents. The toxicity of metals such as lead, mercury and thallium makes them less desirable for in vivo use. Bismuth, barium and tungsten are potential candidates for this specific use, however the X-ray attenuation properties of bismuth and specifically barium is relatively low. Tungsten in the form of tungsten powder is pyrophoric and as such cannot be used as a pharmaceutical.

Although the commercial available soluble iodine containing compounds are considered very safe and are used in more that 20 millions of X-ray examinations annually in the USA, there is still a desire to develop new contrast agents. Such agents should ideally have improved properties over the soluble iodine containing compounds in one or more of the following properties: renal toxicity, viscosity, injection volumes and attenuation/radiation dose.

It has now been found that particles comprising a core of the metallic element tungsten optionally mixed with other metal elements and where the said core is coated with a coating layer such as a polymeric layer or a monomeric layer have surprising and favourable properties as pharmaceuticals, and in particular as contrast agents. The coating layer will passivate the reactive surface of the tungsten particle cores and provide safe nanoparticles with favourable properties.

It should be noted that the terms particles and nanoparticles are used interchangeably when the particles are in the nanometer size, and that core and tungsten core are also used interchangeably in the further document. In the expression pharmaceuticals is also enclosed the particles/nanoparticles which constitute the active principle of the pharmaceutical. Further embodiments are specified in the attached claims and will be outlined in the text.

The compounds of the invention are particles comprising a core and a coating layer. The diameters of the particles are in the nanometer range and they are hence termed nanoparticles. Although the particles can vary in a range from about 1.5 nm to over 20 nm, more preferred from 1.5 to 15 nm, it is frequently preferred that they are excreted by the kidneys. The particle size should therefore preferably be below the kidney threshold of about 6 to 7 nm (Kobayashi, H.; Brechbiel, M. W. Molecular Imaging 2, 1 (2003)), and preferably the particle size should be from 1.5 nm to 7 nm or more preferable from 2 to 6 nm.

The core of the particle contains tungsten in its metallic form or tungsten in mixture with other suitable metallic elements. Preferably the tungsten content is between 20 and 100 weight %, more preferred between 50 and 100%, and even more preferred of 85 to 100 weight % and particularly preferred between 95 and 100 weight %. Cores of about 100% tungsten are generally preferred.

Introducing other metallic elements in the tungsten core can provide improved properties to the core e.g. improve the stability, monodispersity, the synthesis and/or the rate of formation of the metal core. Preferably 5 to 15 weight % of rhenium, iridium, niobium, tantalum or molybdenum either as a single element or as mixtures of elements are feasible additives, most preferred are rhenium and iridium. All these elements are miscible with tungsten and small amounts of rhenium and/or iridium improve the low temperature plasticity of the metallic core.

It is important that the metallic core which provides the attenuating properties to the particles is of a sufficient size with regard to this property taking into consideration the preferred total size of the nanoparticle. The core must hence contain as optimal amount as possible of metal atoms to provide the desired attenuating properties. When the core consist of about 100 weight % tungsten metal, the core should contain from 15 to 5000 tungsten atoms, preferably between 100 and 3000 tungsten atoms and more preferred between 200 and 2500 tungsten atoms. Assuming that the tungsten atoms are packed in body centered cubic crystals, one core of tungsten atoms counting 15 atoms will have a core diameter of about 0.6 nm, 100 tungsten atoms will have a diameter of 1.5 nm, 1500 tungsten atoms will have a diameter of about 4.2 nm whereas a core size of 5 nm will contain about 2500 tungsten atoms and a core containing 5000 tungsten atoms will have a diameter of about 6.5 nm.

Since the tungsten containing core is reactive to a greater or lesser extent, the metallic core must be coated in order to passivate the reactive surface. The properties of the coating should provide a protection to the metallic core such that the core does not react e.g. ignite when exposed to air or react when formulated for in vivo use or react in the in vivo environment. Preferably the coating should maintain its properties until the particles are excreted from the body to which they are administered to such degree that the tungsten surface of the core does not become reactive. The coating should also provide nanoparticles that have a suitable short half-life in vivo. If the nanoparticles contain targeting moieties, the half-life of the particles could be prolonged but it is necessary that the half-life is acceptable taking the toxicity into consideration. It is therefore important that the coating is such that the particles have a low tendency to form aggregates, particularly in vivo. At the same time the coating must be relatively thin in order to provide sufficiently small particles, preferably particles of a size under the kidney threshold of about 6 to 7 nm, although larger particles are also useful for the purpose. The binding between the metal core and the coating should also be sufficiently strong to avoid disintegration between the metallic core and the coating.

The water solubility of the nanoparticles must be high when the pharmaceutical is formulated for parenteral administration, e.g. for injection into a vein or an artery.

The viscosity of the formulated pharmaceutical should also be low enough such that the pharmaceutical can be easily administered. The viscosity is an important factor for pharmaceuticals for parenteral administration. For pharmaceuticals administered via an external void of the body the viscosity is of less importance. The volume fraction of the contrast agent lopamidol in an aqueous solution at 350 mg iodine/ml is 0.26 and the viscosity is 7.6 mPas at 37° C. Assuming that we can use the same volume fraction φ=0.26 for the nanoparticles according to the invention, where the viscosity of the solvent η=0.653 10⁻³ Pas for water at 37° C., the viscosity η of such solution at 37° C. would then be:

$$η = {η}_{0} exp [ \frac{5}{2} ϕ / ( 1 - 1.43 ϕ ) ] \approx 1.84 m Pas \qquad ( I )$$

(see “The viscosity of a concentrated suspension of spherical particles” Mooney, M. J. Colloid. Sci. vol. 6, page 162, (1951)). This viscosity is very low for such a high concentration of particles and relies on the assumption that it is a solution of rigid spheres. This viscosity is also low compared to the viscosity of iodinated X-ray contrast agents.

Metallic tungsten has a relatively high X-ray attenuation value, low toxicity and is available at an acceptable price.

The osmolality of the formulated pharmaceutical is an additional important factor having impact on the toxicity of the product. The osmolality of a solution is determined by the number of dissolved particles per unit of the solvent, usually water. Formulations of high osmolality tend to exert more severe adverse effect in particular arising from intravenous and intra-arterial injections. Formulations of high osmolality cause transport of water across semipermeable membranes resulting in undesired physiological effects. The formulations should therefore ideally be essentially isoosmolal, however slightly hyperosmolal or hypoosmolal formulations are acceptable.

It has been found that specific forms of coatings will fulfil the properties discussed such as to provide nanoparticles comprising the core and the coating that can be used as pharmaceuticals, in particular as contrast agents in medical imaging such as X-ray contrast agents.

In a first embodiment, nanoparticles comprising a metal core coated by a charged coating are provided. By “charge” is meant chemical entities with negative or positive charged groups. The charged coating contains up to 50 charges per nanoparticle, preferably up to 40 charges per nanoparticle, even more preferred up to 25 charges per nanoparticle. Each nanoparticle should not contain less that 4 charges, preferably not less that 8 charges per particle. The number of charges will depend upon the size of the metallic core and also the size of the coated nanoparticle. Coating comprising charged groups with either negative or positive charges will provide particles that repel each other when in solution, and formation of nanoparticle clusters is thereby substantially or partially avoided. Avoiding formation of clusters of the coated particles enhance the solubility of the particles. Further the viscosity of the particle formulation will be kept in a preferred range.

On the other hand the formulation of charged particles will comprise neutralising counter ions and this will lead to a rise in the osmolality. However, since the nanoparticles contain a large number of tungsten atoms it is possible to achieve solutions that are 12 M with respect to tungsten atoms, they would typically only be 60 mM with respect to the number of free particles. Since each charge brings along one counter ion, this gives a large marginal to accept some charges per particle since isoosmotic preparations can be formulated with up to 0.5 M of free particles (including counter ions).

The charged groups must be in their ionic form at the pH of the environment where the compound is used. Most importantly they must be in charged form at physiological pH, in particular at the pH of blood. If the pharmaceutical is meant for non-parenteral administration such as administration through external ducts and voids of the body such as the gastrointestinal tract, the bladder and the uterus, then the coating should have charged form at the specific pH of the target organ.

The coating material can contain groups of positive or negative charges. Anionic groups exerting negative charges can be a wide variety of groups known to the skilled artisan. Of particular importance are acidic groups such as carboxylic acid groups, sulphonic acid groups, phosphoric acids groups and also acidic heterocyclic groups such as tetrazoles or 5-hydroxyisooxazoles. Cationic groups are likewise feasible for the purpose and a wide variety of groups are available. Basic amino, amidine and guanidine groups can be used, as well as quatenary ammonium or phosphonium groups.

The coating layer can comprise material of polymeric or monomeric material. The monomeric material coating should preferably comprise a hydrophilic layer of non-metallic material comprising at least a fraction of molecules that are hydrophilic and preferably each molecule should have at least one hydrophilic group. The coating should at the same time cover the core surface (e.g. the tungsten core surface) densely enough to passivate it. The passivation takes place on the surface of the core where there is an electron transfer between the metal coordination group and the surface of the core. Examples of metal coordinating groups are groups A in the formula Aₙ-Lₒ-Mₚ below. In a preferred aspect the coating is a mono-layer coating meaning that the thickness of the coating is only one single molecule. Monomeric coatings have the benefit that the coating layer can be made thin and with well defined properties. The efficacy of the nanoparticles depends on that the tungsten core of nanoparticle constitutes the highest possible fraction of the particle. At the same time the total diameter of the particle should be small, most preferable below about 6 to 7 nm which is the kidney excretion threshold for parenteral use. The oriented mono molecular layer also provides improved control over solubility and toxicity since there will be a well defined outer end of the molecule where the hydrophilic groups which function as solubilizing groups and the charged groups can be placed, with another end of the molecule facing and binding to the metal.

In a preferred aspect of the invention the mono-layer coating is built according to the general formula Aₙ-Lₒ-Mₚ, where A is one or more metal coordinating groups preferably selected from Table 1, L is absent or present, and when present is one or more linking groups preferably selected from Table 2, and M is one or more charged and hydrophilic groups preferably selected from Table 3. The linking group preferably comprises any number of fragments from Table 2 arranged linearly, branched or in one or more rings. The branching may be towards the A group side to create multidentate coatings or it may branch towards the M group to create a higher degree of hydrophilicity. Branching in both directions is also an option. Linking fragments from Table 2 may be combined to phenyl rings or aromatic or non-aromatic heterocyclic groups. n is any positive integer and preferably from 1 to 10 or more preferably from 1 to 4. o is zero or any positive integer and preferably from 1 to 10 or more preferably from 1 to 2. p is any positive integer and preferably from 1 to 10 or more preferably from 1 to 4. The dotted line for the groups A indicates a bond to the tungsten element, a bond to an H-atom, a bond to the L-group, a bond to another A-group or a bond to the M group when o is zero. The dotted line for the groups L indicates a bond to the A group, a bond to an H-atom, a bond to another L-group or a bond to the M-group. The dotted line for the groups M indicates a bond to the L group, a bond to an H-atom, a bond to another M-group or a bond to the A-group when o is zero.

TABLE 1
Metal coordinating groups A:

| | | |
|---|---|---|
| 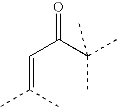 | 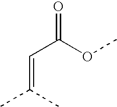 | 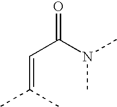 |
| 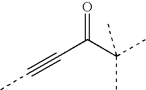 | 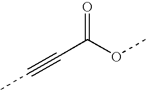 | 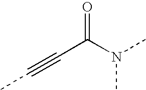 |

| | | | | |
|---|---|---|---|---|
| 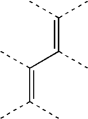 | 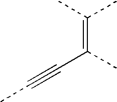 | 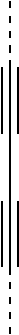 | 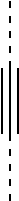 | 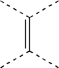 |

| | | |
|---|---|---|
| 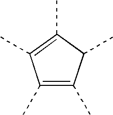 | 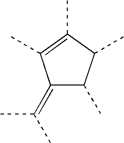 | 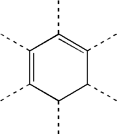 |

| | | | |
|---|---|---|---|
| 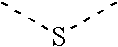 | 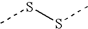 | 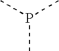 | 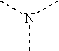 |
| 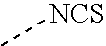 | 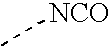 | 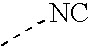 | 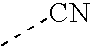 |
| 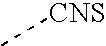 | 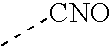 | | |

| | | |
|---|---|---|
| 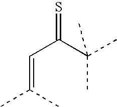 | 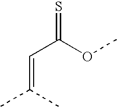 | 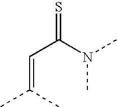 |
| 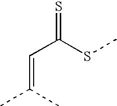 | | |
| 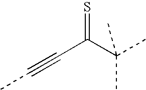 | 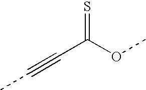 | 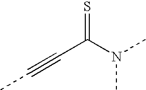 |
| 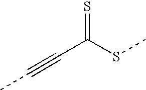 | | |

TABLE 2
Linking groups L:

| | | | |
|---|---|---|---|
| 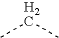 | 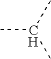 | 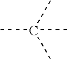 | |
| 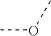 | 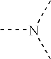 | 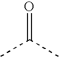 | 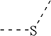 |
| 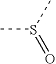 | 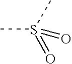 | 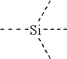 | |

TABLE 3
Hydrophilic groups M:

| | | | |
|---|---|---|---|
| 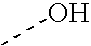 | 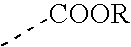 | 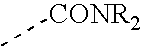 | 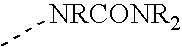 |
| 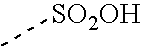 | 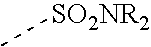 | 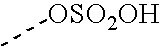 | 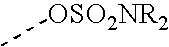 |
| 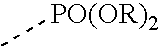 | 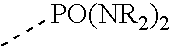 | 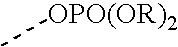 | 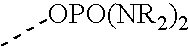 |
| 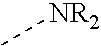 | 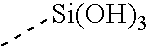 | 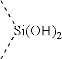 | 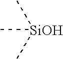 |

The R-groups are independently any group(s) selected from H and a C₁-C₆ alkyl group optionally substituted by one or more —OH groups and where one or more of the C-atoms of the C₁-C₆ alkyl group may be replaced by an ether group.

The polymeric material coating comprises a layer of any polymeric material suitable for pharmaceutical use containing a minimum number of charged groups per nanoparticle and being hydrophilic. The coating should cover the tungsten surface densely enough to passivate it. The polymeric surface layer can be covalently bound to the metallic core surface or adsorbed and held by non-covalent forces. As described above for the monomeric coating, it is preferred that the coating layer is as thin as possible and at the same time providing the necessary passivation of the tungsten core surface. The polymer can be a natural or synthetic homopolymer or copolymer. Numerous polymers are available for the purpose and the skilled artisan will be able to choose suitable polymers known from the state of art. Useful classes of polymers comprises polyethers (e.g PEG and optionally branched), polyacetals, polyvinylalchohols and polar derivatives thereof, polyesters, polycarbonates, polyamides including aliphatic and aromatic polyamides and polypeptides, classes of carbohydrates such as starch and cellulose, polycyanoacrylates and polycyanometacrylates, provided that the polymers contain a minimum of charged groups and most preferable also are hydrophilic. Polymers made of acrylic acid monomers are specifically preferred. In order to obtain a layer with a controlled and suitable number of charged groups, copolymers are also preferred wherein the copolymer can contain 2 or more momomeric entities or blocks. At least one of the monomers shall provide charged groups to the polymer coating. The charge increases the water-solubility and reduces the risk of particle aggregation, but also increases the osmolarity of the particles. Thus, the number of charge carrying groups should be kept at a minimum. In preparations, a neutral monomer combined with a charged monomer in molar ratios below 20:1, preferably from 10:1 to 10:1.5 can provide a polymer with a suitable number of charges for nanoparticles of a diameter from 2 to 6 nm. Possibly, this ratio could be increased even further. Use of monomer F forms a cross-linked polymer.
Examples of suitable monomers to be used to form the polymer coating are:

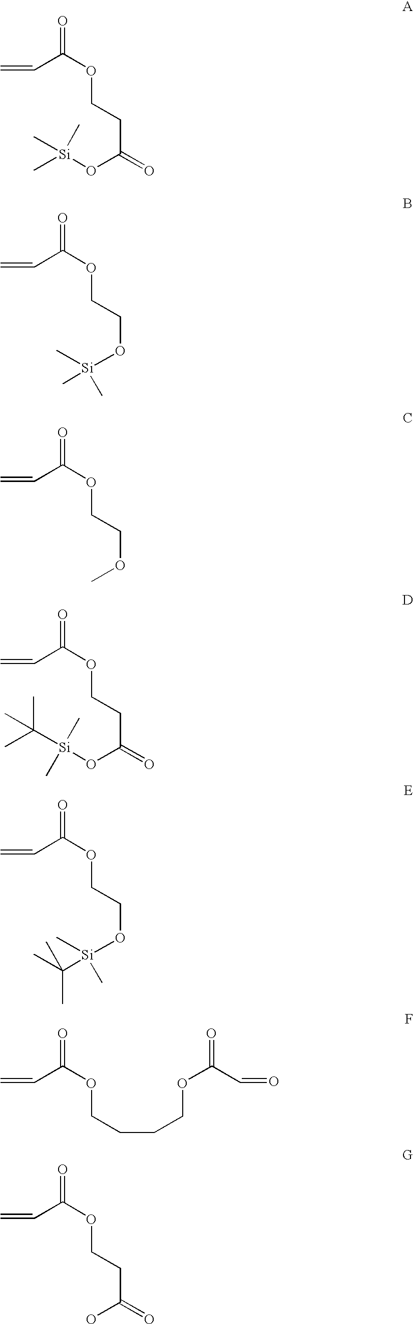

Generally, the polymer coated nanoparticles are prepared by thermally decomposing a source of tungsten (0), e.g. tungsten hexacarbonyl, W(CO)₆, in a high-boiling, dried and deoxygenated solvent in the presence of one or more of the monomers. A thermally induced polymerization of the monomers takes place, covering the tungsten particles formed from the decomposition, with a polymeric coating. When the monomers comprises silylether-protected polar groups (—OH, —COOH) the protecting groups are cleaved in aqueous solution to yield the hydrophilic polymer coated particles.

Dry solvents should generally be used. Hygroscopic solvents (diglyme, triglyme) should be percolated through alumina and stored over molecular sieves. All solvents should be deoxygenated by letting a stream of argon bubble through the solvent for 25-30 minutes before they are used in the reactions. The choice of solvent for this process is critical since there are several criteria to be fulfilled. One is the ability to dissolve the starting materials and at the same time keep the final polymer coated particles in solution. The polyethers di- and triglyme are particularly useful here. The high boiling point of triglyme in particular, will allow the temperature to reach the level where the last carbon monoxide molecules leave the particles. Other useful solvents would be diphenyl ether and other inert high-boiling aromatic compounds. Also trioctyl phosphine oxide (and other alkyl analogs), trioctyl phosphine (and other alkyl analogs), high boiling amides and esters would be useful.

Another important process parameter is the ability to control the tendency of W(CO)₆ to sublimate out of the reaction mixture. This can be achieved by mixing in a small fraction of a lower boiling solvent to continuously wash back any solid tungsten hexacarbonyl from the condenser or vessel walls. Cyclooctane and n-heptane would be good choices when used in 5 to 15% volume fraction.

For the work-up of the particles, precipitation by the addition of pentane or other low-boiling alkanes would be convenient. A low boiling point solvent is advantageous when the particles are to be dried.

The preparation and work-up procedures are further described in the specific examples.

In a second embodiment the core is coated with a hydrophilic layer not containing charged groups. The coating should preferably be a layer of a monomeric material coating and should comprise a hydrophilic layer of non-metallic molecules comprising at least a fraction of molecules that are hydrophilic and preferably each molecule should have at least one hydrophilic group as described above.

The surface coating may include a targeting moiety such as an antibody, antibody fragment, peptide, lipid, carbohydrate, nucleic acid, a drug or drug fragment or any other molecule that is able to direct the pharmaceutical to a specific organ or structure in the body to be examined. Examples of organs or structures to be targeted are the endoreticular system of the liver and spleen, constituents of clots in the blood stream, constituents of atherosclerotic plaque, tumour markers and macrophages.

Contrast media are frequently administered parentally, e.g. intravenously, intra-arterially or subcutaneously. Contrast media can also be administered orally or via an external duct, e.g. to the gastrointestinal tract, the bladder or the uterus. Suitable carriers are well known in the art and will vary depending on e.g. the administration route. The choice of carriers is within the ability of the skilled artisan. Usually aqueous carriers are used for dissolving or suspending the pharmaceutical, e.g. the contrast agent to produce contrast media. Various aqueous carriers may be used such as water, buffered water, saline, glycine, hyaluronic acid and the like.

It will be possible to formulate solutions containing the nanoparticles of the invention having from about 1.0 to about 4.5 g tungsten/ml solution, more specifically from 1.5 to about 3.0 g tungsten/ml water and most specifically about 2.2 g tungsten/ml water. This corresponds to a content of tungsten of about 12 M. A typical nanoparticle formulation will preferably have between 200 and 2500 tungsten atoms in the core.

For use as pharmaceuticals the tungsten containing nanoparticles must be sterilized, this can be done by techniques well known is the state of art. The particles can be provided in sterile solution or dispersion or alternatively in dry form, e.g. in lyophilized form.

The invention will hereinafter be further illustrated with the non-limiting examples.

Examples 1 to 5 describe production of tungsten cores coated by a monomeric layer, whereas examples 6 to 10 describe charged polymeric coating of tungsten cores. All temperatures are in ° C.

The monomers A to G used in the examples are:

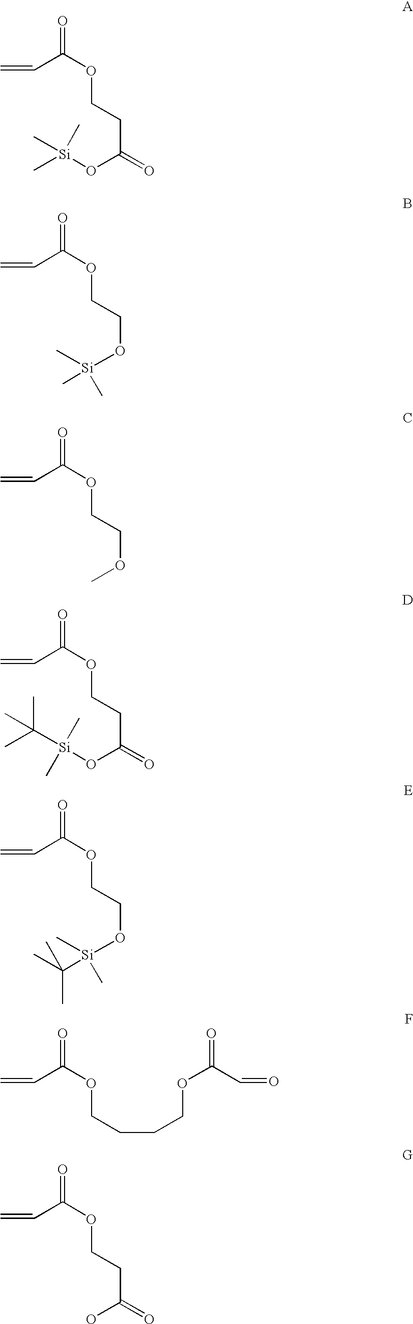

Analysis of the polymer coated particles was done mainly by the means of NMR (¹³C, ¹H), IR, and X-ray fluorescence spectroscopy (XFS). In one case a TEM micrograph was obtained.

In general, broadened ¹H NMR peaks and lack of resonances in the double bond region implied complete polymerisation. The ¹³C NMR spectra showed, in addition to the resonances from the aliphatic part of the polymer, several closely spaced (within 3 ppm) resonances in the carbonyl region. No resonances from residual metal carbonyls were detected by NMR.

The IR spectra showed strong absorptions from the polymer carbonyl groups and, to a varying extent, residual metal carbonyls.

The tungsten content in the particles was determined by X-ray fluorescence spectroscopy.

Particle degradation experiments were performed with UV-Vis spectroscopy (300-800 nm) in deoxygenated tris-glycine buffer solutions.

Electrophoresis experiments, performed in tris-glycine buffer (pH 7.5), showed the negative charge of particles comprising monomers A and D.

A Malvern Zetasizer instrument, using Diffusion Light Scattering (DLS), was used to determine particle size of one of the preparations.

Solubility in water was determined by dissolving the particles in a Tris-glycine buffer (0.1 M, pH 7.5) and freeze-drying the solution. The solubility of the resulting powder was then roughly determined.

### EXAMPLE 1

### Preparation of Tungsten Nanoparticles by Reduction in an Organic Solvent

The reaction is performed under inert gas. A tungsten compound (e.g. WCI₆) and a coating where reactive sites are protected by protection groups are dissolved in an aprotic, water immiscible organic solvent and a soluble reduction agent is added. After the reaction is complete, water and organic solvent are added and the phases are separated. The organic layer is washed with water and evaporated to a small volume. A large excess of ethanol/water is added and the solids are allowed to precipitate. The solids are filtered off and the dissolution, precipitation procedure is repeated once more. The particles are dried in vacuum.

The protecting groups are removed by a suitable procedure. If necessary the solution is desalinated by dialysis, size exclusion chromatography, or some other suitable technique. The final product is typically obtained by freeze drying.

### EXAMPLE 2

### Preparation of Tungsten Nanoparticles by Reduction in Water

A water-soluble tungsten compound e.g. sodium tungstate and a coating molecule are dissolved in deoxygenated water under an inert atmosphere. The pH is adjusted to a desired value. This solution is then added to a vigorously stirred solution of reducing agent in degassed water. After complete reduction, the solution is reduced in volume, desalinated by dialysis and then freeze dried to give the final product.

### EXAMPLE 3

### Preparation of Tungsten Nanoparticles by Reduction in Inverse Micelles

An aqueous solution of a water soluble tungsten compound, e.g. sodium tungstate adjusted to a desired pH is introduced as the aqueous phase into an inverse micelle in an organic solvent by the addition of a large fraction of surfactant. A similar inverse micelle formulation of an aqueous reduction agent is also made. The tungsten containing liquid is added to the reduction agent. Coating molecules are added. After equilibration, water is added to break the emulsion. The aqueous phase is collected and the organic phase is washed with two more portions of water. The collected aqueous phases are reduced in volume and desalinated by dialysis. The aqueous solution is then freeze dried to give the final product.

### EXAMPLE 4

### Preparation of Tungsten Nanoparticles by Decomposition of a Tungsten (0) Complex

A thermally labile W(0) complex, e.g. W(CO)₆ is decomposed in an inert, high boiling solvent, e.g. cyclooctane, in the presence of coating molecules where reactive sites are protected by protection groups, e.g. hexylacrylate. After completed reaction, a polar solvent such as ethanol is added; the black powder is filtered of and washed.

The protecting groups are removed by e.g. hydrolysis or other suitable procedures. The solution is reduced in volume and desalinated. The aqueous solution is then freeze dried to give the final product.

### EXAMPLE 5

### Synthesis of N,N-bis(2-hydroxyethyl)acrylate-coated Tungsten Nano-particles

The reaction is carried out under air free conditions. Tungsten hexacarbonyl and N,N-bis(2-dimethyl-tert-butylsilyloxyethyl)acrylate are dissolved in cyclooctane and heated to reflux for 12 hours. Most of the solvent is removed in vacuum and the black residue is washed three times with methanol.

The protecting groups are removed by hydrolysis in 10% aqueous formic acid. The liquids are evaporated, the residue dissolved in water and taken to dryness again. The product is formed as a black powder, wherein the coating layer comprises the molecule H₂C═C—CO—N (CH₂—CH₂OH)₂.

### EXAMPLE 6

### Preparation of a Polymer Coated Tungsten Nanoparticle Comprising Monomers B and C

In a round-bottomed flask fitted with a magnetic stirrer and a condenser was put: Tungsten hexacarbonyl W(CO)₆ (500 mg, 1.4 mmol), ethyleneglycol methylether acrylate (C) (390 mg, 3.0 mmol), and trimethylsilyl protected 2-carboxyethyl acrylate (B) (120 mg, 0.55 mmol). The condenser was fitted with a septum and several vacuum / argon cycles were applied to deairiate the flask and condenser. Deairiated diglyme (30 ml) and heptane (2 ml) were added through the septum with a syringe. The reaction mixture was heated to reflux under an argon atmosphere. After 3 h, the reaction mixture, now a black solution with small amounts of black precipitate, was cooled to room temperature, poured on deairiated pentane (60 ml) and centrifuged. The precipitate was washed with pentane and dried in vacuum.

Yield: 430 mg dark grey powder. X-ray fluorescence spectroscopy analysis showed the tungsten content to be around 60%. Comments: The heptane is needed to prevent tungsten hexacarbonyl sublimation deposits in the condenser. The trimethyl silyl protection group is spontaneously cleaved in aqueous solutions, yielding the preferred carboxylate G.

The particles have a core of crystalline tungsten covered by a thin coating of co-polymerized C and B. The particles are between 4 and 5 nm.

### EXAMPLE 7

### Preparation and Analysis of Polymer Coated Tungsten Nanoparticles Comprising Monomers B and D

Tungsten hexacarbonyl (440 mg, 1.2 mmol), monomer B (970 mg, 5.0 mmol) and monomer D (300 mg, 1.1 mmol) were put in a glass flask equipped with a condenser and a magnetic stirrer. The flask and condenser were subjected to several vacuum/ argon cycles leaving an argon atmosphere. Cyclooctane (30 ml) was added with a syringe through a septum at the top of the condenser. The reaction solution was stirred and heated to reflux for 18 h. During the first hours, the solution slowly darkened, eventually becoming black (as strong coffee). After completed reaction time, the solution was cooled to room temperature and poured on pentane (50 ml). The resulting slurry was centrifuged and the precipitate was washed with pentane and dried in vacuum.

Yield: 400 mg dark powder Analysis ¹H NMR: broadened resonances appeared at (ppm) 4.3, 4.1, 3.8, 3.5, 2.8, 2.7-2.2,1.8-1.2, 0.8, 0.1. IR: 1939 w, 1852 w, 1731 vs, 1560 m. XFS: 57% W Solubility in water: >500 mg/ml.

### EXAMPLE 8

### Preparation and Analysis of Polymer Coated Tungsten Nanoparticles Comprising Monomers A and C

Tungsten hexacarbonyl (500 mg, 1.4 mmol), monomer A (120 mg, 0.55 mmol) and monomer C (390 mg, 3.0 mmol) were added to the glass flask following the procedure of example 7. Diglyme (30 ml) and heptane (2 ml) were added through the condenser. The reaction solution was stirred and then heated to reflux for 3 h. Yield: 410 mg dark powder.

Analysis: ¹H NMR: broadened resonances appeared at (ppm) 4.1, 3.5, 3.2, 2.5-2.2,1.9-1.3. IR: 1995 w, 1894 w, 1727 vs, 1540 s. XFS: 55% W. TEM: a micrograph showing particle cores in the size of 3-4 nm was obtained. Degradation experiment: an exponential decrease in absorption over the whole spectrum (300-800 nm). At most, the absorption decreased 22% in 4.3 h (at 350 nm). Electrophoresis experiment: movement of the particles implied negative charge.

### EXAMPLE 9

### Preparation and Analysis of Polymer Coated Tungsten Nanoparticles Comprising Monomer E

Tungsten hexacarbonyl (2.3 g, 6.5 mmol)m and monomer E (7.6 g, 32 mmol) were added to the glass flask following the procedure of example 7. Cyclooctane (100 ml) were added through the condenser. The reaction solution was stirred and then heated to reflux for 60 h.

Analysis: Size of particles was determined by Dynamic Light Scattering. 99% of the total particle volume belonged to particles having a size between 5.8-7.8 nm.

### EXAMPLE 10

### Preparation and Analysis of Polymer Coated Tungsten Nanoparticles Comprising Monomer A, C and F

Tungsten hexacarbonyl (1.0 g, 2.8 mmol), triglyme (45 ml) and heptane (3 ml) were put in a glass flask equipped with a condenser and a magnetic stirrer. The flask and condenser were subjected to several vacuum/argon cycles leaving an argon atmosphere. The slurry was heated and stirred until dissolution. The solution was then heated to 160° C. after which a mixture of monomer C (1.8 g, 14 mmol), monomer A (280 mg, 1.3 mmol) and monomer F (280 mg, 1.4 mmol) was added with a syringe through a septum. The solution was stirred at 165-170° C. for 3 h. After completed reaction time, the solution was cooled to room temperature and poured on pentane (50 ml). The resulting slurry was centrifuged and the precipitate was washed with pentane and dried in vacuum. Yield: 800 mg dark powder.

Analysis ¹H NMR: broadened resonances appeared at (ppm) 4.2, 3.5, 3.3, 2.3, 2.0-1.4. IR: 1921 w, 1825 w, 1727vs, 1534m. XFS: 47% W.

What is claimed is:

1. A particle comprising a core of the metallic element tungsten optionally together with other metallic elements wherein said core is coated with a coating layer.

2. A particle as claimed in claim 1 wherein the diameter is in the range of about 1.5 to about 20 nm.

3. A particle as claimed in claim 1 wherein the diameter is in the range of 1.5 to 15 nm.

4. A particle as claimed in claim 1 wherein the diameter is in the range of 1.5 to 7 nm.

5. A particle as claimed in claim 1 wherein the diameter is in the range of 2 to 6 nm.

6. A particle as claimed in claim 1 wherein the core of the particle has a tungsten content of 20 to 100 weight % of metallic tungsten.

7. A particle as claimed in claim 1 wherein the core of the particle has a tungsten content of 50 to 100 weight % of metallic tungsten.

8. A particle as claimed in claim 1 wherein the core of the particle has a tungsten content of 85 to 100 weight % of metallic tungsten.

9. A particle as claimed in claim 1 wherein the core of the particle has a tungsten content of 95 to 100 weight % of metallic tungsten.

10. A particle as claimed in claim 1 wherein the core of the particles has a tungsten content of about 100 weight % of metallic tungsten.

11. A particle as claimed in claim 1 wherein the core of the particle comprises metallic tungsten and one or more of the elements rhenium, iridium, niobium, tantalum or molybdenum in their metallic form.

12. A particle as claimed in claim 1 wherein the coating layer comprises a charged coating layer.

13. A particle as claimed in claim 12 wherein the charged coating layer provides a net positive or negative charge at the pH of the environment where the particle is administered.

14. A particle as claimed in claim 12 wherein the charged coating layer provides a negative charge at the pH of the environment where the particle is administered.

15. A particle as claimed in claim 12 wherein the charged coating layer provides the net negative charge of acidic groups such as carboxylic acid groups, sulphonic acid groups, phosphoric acid groups and acidic heterocyclic groups.

16. A particle as claimed in claim 12 wherein the charged coating layer provides the net positive charge of basic amino, amidine, guanidine, quaternary ammonium and phosphonium groups.

17. A particle as claimed in claim 12 wherein the charged coating layer comprises up to 50 charges per particle.

18. A particle as claimed in claim 12 wherein the charged coating layer comprises up to 40 charges per particle.

19. A pharmaceutical as claimed in claim 12 wherein the charged coating layer comprises up to 25 charges per particle.

20. A particle as claimed in claim 12 wherein the charged coating layer comprises at least 8 charges per particle.

21. A particle as claimed in claim 12 wherein the charged coating layer comprises at least 4 charges per particle.

22. A particle as claimed in claim 12 wherein the layer comprises a polymeric layer with charged groups.

23. A particle as claimed in claim 22 wherein the polymeric layer comprises a hydrophilic polymer.

24. A particle as claimed in claim 22 wherein the polymeric layer comprises a homopolymer.

25. A particle as claimed in claim 22 wherein the polymeric layer comprises a copolymer.

26. A particle as claimed in claim 22 wherein the polymeric layer is formed from acrylic acid monomers.

27. A particle as claimed in claim 22 wherein the polymeric layer is formed from at least one monomer containing a charged group.

28. A particle as claimed in claim 22 wherein the polymeric layer is formed from at least one neutral monomer.

29. A particle as claimed in claim 22 wherein the molar ratio between the neutral monomer and the charged monomer as is below 20:1.

30. A particle as claimed in claim 29 wherein the molar ratio between the neutral monomer and the charged monomer is between 10:1 and 10:1.5.

31. A particle as claimed in claim 1 wherein the layer comprises a monomeric layer.

32. A particle as claimed in claim 31 wherein the monomeric layer comprises a hydrophilic monomeric layer.

33. A particle as claimed in claim 32 wherein said hydrophilic layer comprises at least a fraction of molecules that are hydrophilic.

34. A particle as claimed in claim 31 wherein said hydrophilic layer comprises molecules that each has at least one hydrophilic group.

35. A particle as claimed in claim 1 wherein said core is coated with a mono-layer coating.

36. A particle as claimed in claim 35 wherein said mono-layer coating comprises compounds of formula Aₙ-Lₒ-Mₚ, where A is one or more tungsten coordinating groups, L is absent or is one or more linking groups and M is one or more hydrophilic groups, n and p are positive integers and o is zero or a positive integer.

37. A particle as claimed in claim 31 wherein the monomeric layer comprises a charged coating layer.

38. A particle as claimed in claim 37 wherein the charged coating layer provides a net positive or negative charge at the pH of the environment where the particle is administered.

39. A pharmaceutical comprising particles of claim 1 optionally together with a pharmaceutically acceptable solvent or excipient.

40. A diagnostic agent comprising particle as claimed in claim 1 optionally together with a solvent or excipient.

41. An X-ray contrast agent comprising a particle as claimed in claim 1 optionally together with a solvent or excipient.

42. A particle as claimed in claim 1 wherein the particle comprises in vivo contrast agents.

43. A particle as claimed in claim 1 wherein the particle comprises X-ray contrast agents.

44. A method of diagnosis comprising administration of particles of claim 1 to a human or animal body, examining the body with a diagnostic device and compiling data from the examination.

45. A method of imaging, specifically X-ray imaging comprising administration of particles of claim 1 to a human or animal body, imaging the body with an imaging device, compiling data from the examination and optionally analysing the data.

46. A process for the preparation of particles of claim 1 comprising decomposing a source of tungsten (0) in a high boiling, dried and deoxygenated solvent in the presence of one or more monomers and thereby effecting a thermally induced polymerization of the monomers.

47. A process as claimed in claim 46 wherein the source of tungsten (0) is tungsten hexacarbonyl (W(CO)₆).

48. A process as claimed in claim 46 wherein the solvent comprises di- and triglyme, diphenyl ether, trialkyl phosphine oxide and trialkyl phosphine.

49. A process as claimed in claim 48 wherein the solvent comprises trioctyl phosphine oxide and triaoctyl phosphine.

50. A process as claimed in claim 46 wherein the high boiling, dried and deoxygenated solvent further comprises a fraction of a lower boiling solvent.

51. A process as claimed in claim 50 wherein the fraction of a lower boiling solvent comprises between 5 to 15 volume % of cyclooctane and/or n-heptane.

52. A process as claimed in claim 46 further comprising work-up of the formed particles from a low-boiling alkane, specifically from pentane.

53. A process as claimed in claim 46 wherein one or more of the monomers comprises silylether-protected polar groups and where the protecting groups are cleaved off in aqueous solution to yield hydrophilic polymer coated particles.

54. A particle as claimed in claim 37 wherein the charged coating layer provides a negative charge at the pH of the environment whre the particle is administered.

55. A particle as claimed in claim 37 wherein the charged coating layer provides the net negative charge of acidic groups such as carboxylic acid groups, sulphonic acid groups,phosphoric acid groups and acidic heterocyclic groups.

56. A particle as claimed in claim 37 wherein the charged coating layer provides the net positive charge of basic amino, amidine, guanidine, quaternary ammonium and phosphonium groups.

57. A particle as claimed in claim 37 wherein the charged coating layer comprises up to 50 charges per particle.

58. A particle as claimed in claim 37 wherein the charged coating layer comprises up to 40 charges per particle.

59. A particle as claimed in claim 37 wherein the charged coating layer comprises up to 25 charges per particle.

60. A particle as claimed in claim 37 wherein the charged coating layer comprises at least 8 charges per particle.

61. A particle as claimed in claim 37 wherein the charged coating layer comprises at least 4 charges per particle.
